(19) United States
(12) Patent Application Publication
(10) Pub. No.: US 2019/0142320 A1
(43) Pub. Date: May 16, 2019

(54) FLUID SAMPLING DEVICE AND METHOD FOR USING SAME

(71) Applicant: BACALA INNOVATIONS PTY LTD, Warrandyte (AU)

(72) Inventors: Luke Sammartino, Warrandyte, Victoria (AU); James Daniel John, Preston, Victoria (AU); Benjamin John Druce, Box Hill, Victoria (AU); Say Leong Melvin Choo, Clayton, Victoria (AU)

(21) Appl. No.: 16/098,505

(22) PCT Filed: May 4, 2017
(86) PCT No.: PCT/AU2017/050407

§ 371 Date: Nov. 2, 2018

(30) Foreign Application Priority Data
May 4, 2016 (AU) 2016901638

(51) Int. Cl. A61B 5/153; A61B 5/15

(52) U.S. Cl. CPC A61B 5/1535; A61B 5/15003; A61B 5/150343; A61B 5/150572; A61B 5/150389; A61B 5/150503; A61B 5/150732; A61B 5/150259

(57) ABSTRACT

A device for sampling fluid from a subject includes a body having a fluid inflow end and a fluid outflow end, and a needle adapted to penetrate the subject to sample fluid. The needle is in fluid communication with the body at the fluid inflow end. The body has a body portion at the fluid outflow end that is couplable with a collecting receptacle. The coupling is such that an interior of the collecting receptacle remains in fluid communication with the ambient environment to permit the fluid to be passively collected into the collecting receptacle during fluid sampling. A method for sampling fluid from a subject with a sampling device is provided. A collecting receptacle releasably couplable with a device for sampling fluid from a subject is also provided. A kit including a sampling device and one or more collecting receptacles is also provided.

This application claims priority from Australian Provisional Patent Application No. 2016901638 filed on 4 May 2016, the contents of which are to be taken as incorporated herein by this reference.

### TECHNICAL FIELD

The present invention relates to a device and method for sampling fluid from a subject, and more particularly but not exclusively to improved blood sampling from a subject with small or fragile vessels.

### BACKGROUND OF INVENTION

Techniques are known which enable fluid sampling from subjects, including venipuncture, which provides intravenous access for blood sampling from veins. The traditional method for blood collection is the syringe and needle technique, which involves inserting the needle into a vein, observing “flash-back” of blood to indicate correct positioning of the needle in a vein, and drawing up the syringe plunger to collect blood. Advantageously, the syringe and needle technique is simple and drawing the blood is a manual operation. The operator controls the amount of suction applied to the vein. In addition, this approach is compatible for drawing blood from many different patients due to the variety of needle and syringe sizes available.

However, there are particular subsets of patients for which the syringe and needle technique consistently achieves poor results, or is not possible. One subset includes neonates and young children with small veins for whom there is increased difficulty in correctly positioning the needle into a vein due to movement of the patient. Another subset includes elderly, oncology, burns or obese patients with small or fragile veins, which may collapse under the amount of suction applied by a syringe. In both cases, the syringe and needle technique can hinder blood sampling, requiring additional attempts to obtain a sample at the patient's discomfort.

The “vacutainer system” has been developed as another method to sample blood intravenously. The method uses a needle attached to a tube holder, with a vacuum pressurised sleeve and a sampling tube called a vacutainer. The tubes are designed to provide a vacuum that automatically aspirates blood into the tube under negative pressure once engaged with the tube holder. Advantageously, the vacutainer system is simple to use and enables multiple samples to be taken from a single needle insertion. However, the vacutainer system suffers similar drawbacks to the syringe and needle technique. As a result of suction being applied to the vessel through negative pressure, there may be an increased risk of vessel collapse for patients with small or fragile veins. Furthermore, it is not possible to observe flash-back with the vacutainer system due to the presence of the sleeve, contributing to difficulty in correctly positioning the needle into the vein.

To address these issues with blood sampling for neonates and young children, syringes with a butterfly needle have been developed. Unfortunately, there are no other techniques which have been developed for adults including elderly, oncology, burns or obese patients. Butterfly needles are much smaller than the usual needle size and are connected to the syringe via a section of thin tubing. The syringe with a butterfly needle is less painful to insert into the veins and reduces the risk of vessel collapse for neonates and young children with small or fragile veins. In addition, flash-back is visible in the tubing to guide correct needle placement and the syringe is still manually controlled via suction. However, the butterfly needle is hazardous as while blood flows through the tubing, the blood cools significantly and may clot to the extent that blood flow is impeded. Once this occurs, it is necessary to re-stick the patient, resulting in additional attempts at blood sampling. Moreover, due to the thin diameter of the tubing and cooling of the blood, the blood sample may hemolyse during collection, potentially giving rise to invalid samples.

Another technique for blood sampling from neonates and young children includes the broken-needle technique, although it is dangerous and rarely used. This technique includes breaking off the hub of a needle, inserting the “broken” needle into a vein, observing flash-back of the blood and allowing the blood to drip into a collection tube. Beneficially, the blood is sampled without suction which is gentler than the needle and syringe and vacutainer techniques, and reduces risk of vessel collapse for patients with fragile veins. However, the broken-needle can slip up into the vessel, travel through the circulation and damage organs or tissues in the body. Therefore, the broken-needle technique is not recommended for use in general practice.

Accordingly, there is a need for an improved device and method for sampling blood and other fluids from a patient with small or fragile veins. It would be desirable for the device and method to sample blood or other fluids safely, with little risk of vessel collapse or needle migration into the vessel, and to obtain samples more consistently on the first attempt.

A reference herein to a patent document or other matter which is given as prior art is not to be taken as an admission or a suggestion that the document or matter was known or that the information it contains was part of the common general knowledge as at the priority date of any of the claims.

### SUMMARY OF INVENTION

In a first aspect, the present invention provides a device for sampling fluid from a subject, the device including: a body having a fluid inflow end and a fluid outflow end; and a needle adapted to penetrate the subject to sample fluid, wherein the needle is in fluid communication with the body at the fluid inflow end, wherein the body has a body portion at the fluid outflow end that is couplable with a collecting receptacle, and wherein the body portion that is couplable with the collecting receptacle is configured such that an interior of the collecting receptacle remains in fluid communication with the ambient environment to permit fluid to be passively collected into the collecting receptacle during fluid sampling.

In some embodiments, the body portion is couplable with the collecting receptacle such that it does not entirely occlude an opening of the collecting receptacle. The body portion may include an elongate structure for coupling with the collecting receptacle. The elongate structure may include one or more longitudinally extending grooves that, when coupled with the collecting receptacle each form a gap between the elongate structure and coupled collecting receptacle for fluid communication.

The grooves of the elongate structure may be arranged to mitigate simultaneous occlusion of gaps between the elongate structure and the coupled collecting receptacle with sampled fluid from the subject during fluid collection. Preferably, the one or more longitudinally extending grooves are spaced apart circumferentially around the elongate structure to form correspondingly arranged channels between the elongate structure and the collecting receptacle when coupled.

In some embodiments, the body includes a transparent portion for observing fluid flash-back into the body during fluid sampling. Observation of fluid flash-back is useful for confirming good location of the needle for fluid access. The transparent portion may also be shaped to form a lens that magnifies viewing of the fluid flash-back. In some embodiments, the body includes a lumen between the fluid inflow end and the fluid outflow end for guiding the sampled fluid towards the fluid outflow end for collection in the collecting receptacle. The lumen may be visible through the transparent portion so that fluid flash-back into the lumen at the fluid inflow end is observable during fluid sampling. Preferably, the device is substantially transparent for observing fluid flow through the device during fluid sampling.

In some embodiments, at least part of the collecting receptacle is compliant to allow the sampled fluid to be expelled therefrom by squeezing or compressing. The body may include a protective frame configured to protect the compliant part of the collecting receptacle when the body and collecting receptacle are coupled together. The frame may include a slot for inspecting the fluid level in the collecting receptacle during fluid collection. The collecting receptacle may also include an indicator to allow the amount of fluid collected to be ascertained during fluid collection.

The body may be releasably couplable with the collecting receptacle. One or both of the body and collecting receptacle may include an engagement surface that urges the collecting receptacle and body apart when there is relative rotational movement between the components, thereby decoupling the collecting receptacle and the body. The engagement surface may include a contoured edge on the body and a corresponding contoured edge on a portion of the collecting receptacle, the corresponding contoured edges being configured to rest in abutment when the body and the collecting receptacle are coupled, and to urge apart the body and the collecting receptacle when one is rotated relative to the other, wherein the relative movement between the abutting contoured edges urges the components apart.

In some embodiments, the contoured edge of the collecting receptacle may include at least one winged portion having an edge that is shaped to abut against the contoured edge of the body. The collecting receptacle may be rotated relative to the body by an operator grasping and rotating the winged portion. Preferably, the protective frame includes the contoured edge of the body.

The body may include contoured surfaces adapted to be held by an operator while manipulating the device. The contoured surfaces may be texturized for improved gripping by the operator. Preferably, the contoured surfaces are positioned on opposite sides of the body and assist with correct orientation of the device when in use.

In some embodiments, the body is configured to self-anchor the needle in the subject during fluid sampling. The fluid inflow end of the body may be releasably couplable with the needle, enabling the needle to be replaced and/or interchangeable with different needle sizes. Preferably, the needle size is 21 or 23 gauge, although other needle sizes are possible.

In some embodiments, the device includes a safety mechanism to prevent accidental needle penetration after fluid sampling. The safety mechanism may include a cap that is configured to cover the needle while not in use.

The device may be adapted to sample fluid from a range of anatomical features of the subject, such as but not limited to a blood vessel, a cyst, an abscess and a blister. Fluid sampling from these anatomical features may be required for pathology testing and analysis. The device as described herein is used to sample fluid from human subjects however it may also be suitable for use with animal subjects, such as in veterinary application. The sampled fluid may have a volume in the range of 0.3 mL to 20 mL, depending on the fluid sampling application. In the case of collection of venous blood from paediatric patients, it is preferable that the sampled fluid has a volume in the range of 0.3 mL to 2 mL, and more preferably, a volume of about 1 mL. It will be appreciated that the sampled fluid may have a volume exceeding the range of 0.3 mL to 20 mL, depending on the fluid sampling application, as would be understood by a person skilled in the art.

In another aspect, the present invention provides a collecting receptacle releasably couplable with a device for sampling fluid from a subject, the collecting receptacle being adapted to maintain an interior of the collecting receptacle in fluid communication with the ambient environment while coupled to the fluid sampling device, to permit fluid to be passively collected into the collecting receptacle during fluid sampling.

In some embodiments, the collecting receptacle has an opening and is couplable with the device such that the opening is not fully occluded by the coupling so as to maintain fluid communication between the interior of the collecting receptacle and the ambient environment.

One or both of the device and collecting receptacle may include an engagement surface that urges the collecting receptacle and device apart when there is relative rotational movement between the components, thereby decoupling the collecting receptacle and the device. The engagement surface may include a contoured edge on a portion of the collecting receptacle that is configured to abut against a contoured edge of the device when coupled thereto, and when one of the collecting receptacle and the device is rotated relative to the other of the device and the collecting receptacle, the relative movement between their abutting portions urges the components apart.

In some embodiments, the abutting portion of the collecting receptacle includes at least one winged portion having an edge that is shaped to abut against the contoured edge of the body. The collecting receptacle may be rotatable by an operator grasping and rotating the winged portion.

In some embodiments, at least part of the collecting receptacle is compliant to allow the sampled fluid to be expelled therefrom by squeezing or compressing. The collecting receptacle may also include an indicator to allow the amount of fluid collected to be ascertained during fluid collection. The collecting receptacle may have a volume capacity in the range of 0.3 mL to 20 mL, depending on the fluid sampling application. In the case of collection of venous blood from paediatric patients, it is preferable that the volume capacity is in the range of 0.3 mL to 2 mL, and more preferably, a volume capacity of about 1 mL. It will be appreciated that the volume capacity of the collecting receptacle may exceed the range of 0.3 mL to 20 mL, depending on the fluid sampling application, as would be understood by a person skilled in the art.

In another aspect, the present invention provides a method for sampling fluid from a subject, the method including the steps of: (a) providing a sampling device including a body having a fluid outflow end and a needle at a fluid inflow end of the body; (b) inserting the needle into the subject; (c) sampling fluid by allowing fluid to flow from the subject, through the needle, and into the body through the fluid inflow end for collection at the fluid outflow end in a collecting receptacle; and (d) removing the needle from the subject, wherein the fluid is passively collected into the collecting receptacle during fluid sampling through an arrangement between the fluid outflow end of the body and collecting receptacle that maintains an interior of the collecting receptacle in fluid communication with the ambient environment.

In some embodiments, after providing the sampling device, the method includes the step of coupling the collecting receptacle to the body at the fluid outflow end. The coupling may be such that the interior of the collecting receptacle is in fluid communication with the ambient environment.

In some embodiments, after inserting the needle into the subject, the method includes the step of checking for fluid flash-back in the body to confirm good fluid access. Typically, this is done by visual inspection for the presence of fluid (e.g. blood) within the device, e.g. through a transparent portion of the body of the device during fluid sampling. In some embodiments, most or all of the device is transparent. This enables observation of fluid flash-back and flow of sampled fluid through the device more generally. Observed flow rates may influence the amount of tissue compression applied to the subject (if any) to increase fluid flow. In the absence of fluid flash-back, the method may include the steps of removing the needle from the subject, and repeating the inserting, sampling and removing steps.

The method may further include the step of checking the fluid level in the collecting receptacle during fluid sampling by inspecting an indicator on the collecting receptacle. When the fluid level is sufficient, the method may include the step of decoupling the collecting receptacle by rotating it with respect to the body. After decoupling the collecting receptacle, the method may include the step of squeezing or compressing the decoupled collecting receptacle to expel the sampled fluid into a device, collection vessel or pathology tube. After decoupling the collecting receptacle, the method may further include the steps of coupling a collecting receptacle to the body at the fluid outflow end, and repeating the sampling step.

In some embodiments, the method includes the step of compressing (and releasing) tissue of the subject near the needle to increase fluid flow into the needle. This may be necessary when the rate of flow of sampled fluid is low. In some embodiments, low fluid flow rates can lead to drying or coagulation of sampled fluid, particularly in the case of venous blood sampling, compromising the sample integrity. Ensuring adequate flow by squeezing or compressing the subject's hand, foot or other tissue near the collection site can mitigate this risk. This is particularly useful when the fluid level in the collecting receptacle is considered insufficient. After compressing the tissue to increase fluid flow, the method may include again the step of checking the fluid level in the collecting receptacle.

In some embodiments, the needle is inserted to penetrate the subject to a needle depth sufficient for the sampling device to self-anchor during fluid sampling. The needle may be inserted into a range of anatomical features of the subject, such as but not limited to a blood vessel, a cyst, an abscess and a blister. The sampled fluid may have a volume in the range of 0.3 mL to 20 mL, depending on the fluid sampling application. In the case of collection of venous blood from paediatric patients, it is preferable that the amount of sampled fluid is in the range of 0.3 mL to 2 mL, and more preferably, a volume of about 1 mL. It will be appreciated that the sampled fluid may have a volume exceeding the range of 0.3 mL to 20 mL, depending on the fluid sampling application, as would be understood by a person skilled in the art.

In another aspect, the present invention provides a kit including: (a) a sampling device including a body having a fluid inflow end and a fluid outflow end, and a needle at the fluid inflow end that is adapted to penetrate a subject to sample fluid; and (b) one or more collecting receptacles, wherein the body has a body portion at the fluid outflow end that is couplable with the one or more collecting receptacles and configured such that an interior of the one or more collecting receptacles remains in fluid communication with the ambient environment to permit fluid to be passively collected into the one or more collecting receptacles during fluid sampling.

In some embodiments, the one or more collecting receptacles have a volume capacity selected from a volume in the range of 0.3 mL to 20 mL depending on the fluid sampling application. In the case of collection of venous blood from paediatric patients, it is preferable that the volume capacity is in the range of 0.3 mL to 2 mL, and more preferably, a volume capacity of about 1 mL. It will be appreciated that the volume capacity may exceed the range of 0.3 mL to 20 mL, depending on the fluid sampling application, as would be understood by a person skilled in the art. Preferably, the kit includes two or more collecting receptacles and each collecting receptacle has a different volume capacity. The kit may include collecting receptacles with different volume capacities as required for particular fluid sampling applications.

In some embodiments, the one or more collecting receptacles includes an anticoagulant. The anticoagulant may be selected from one of the group including: sodium heparin, potassium oxalate, ethylene diamine tetraacetic acid (EDTA), sodium citrate, acid citrate dextrose (ACD), sodium polyanethol sulfonate (SPS) and thrombin. Preferably, the kit includes two or more collecting receptacles and each collecting receptacle includes a different anticoagulant. The kit may include collecting receptacles with different anticoagulants as required for particular fluid sampling applications.

In another aspect, the present invention provides a method for sampling fluid from a subject using the kit as described herein.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the provisional claims appended hereto.

FIG. 1 is an isometric view of a device for sampling fluid according to an embodiment of the invention.

FIG. 2 is an exploded view of the components of the device of FIG. 1.

FIG. 3 is a rotated view of the device of FIGS. 1 and 2 showing a collecting receptacle and a body of the device coupled together according to an embodiment of the invention.

FIG. 4 is a top view of the device shown in FIGS. 1 to 3 showing a collecting receptacle and a body of the device coupled together according to an embodiment of the invention.

FIG. 5 is an enlarged view of a body of the device shown in FIGS. 1 to 4.

FIG. 6 is a rotated and tilted view of the body of FIG. 5, showing an elongate structure having longitudinal grooves at a fluid outlet end of the body according to an embodiment of the invention.

FIG. 7 is a top view of the device of FIG. 1 omitting the collecting receptacle to show an elongate structure at the fluid outlet end according to an embodiment of the invention.

FIG. 8 is a sectional view through the line 8-8′ of FIG. 7 showing the cross-section of the elongate structure viewed in the direction away from the fluid inflow end of the body according to an embodiment of the invention.

FIG. 9 is a sectional view through the line 9-9′ of FIG. 4 showing the cross-section of the device viewed in the direction towards the fluid inflow end of the body, and showing gaps formed between the elongate structure and collecting receptacle according to an embodiment of the invention.

FIG. 10 is a top view of the device of FIG. 1 omitting the collecting receptacle to show a transparent portion of a body of the device for viewing fluid flash-back and a lumen of the body to guide sampled fluid according to an embodiment of the invention.

FIG. 11 is an enlarged view of a collecting receptacle according to an embodiment of the invention that is releasably couplable with a device for sampling fluid from a subject.

FIG. 12 is an isometric view of a device for sampling fluid according to an embodiment of the invention, showing a cap covering the needle.

FIG. 13 shows the cap of FIG. 12 decoupled from the body of the device.

FIG. 14 is a flow chart illustrating the steps in a method for sampling fluid from a subject according to an embodiment of the invention.

FIG. 15 is a flow chart illustrating further steps in the method of FIG. 14 relating to coupling a collecting receptacle according to an embodiment of the invention.

FIG. 16 is a flow chart illustrating further steps in the method of FIGS. 14 and 15 relating to checking for fluid-flash back according to an embodiment of the invention.

FIG. 17 is a flow chart illustrating further steps in the method of FIGS. 14 to 16 relating to checking the fluid level according to an embodiment of the invention.

### DETAILED DESCRIPTION

Embodiments of the invention are discussed herein by reference to the drawings which are not to scale and are intended merely to assist with explanation of the invention. The device has utility in venous blood sampling, particularly from subjects with small and/or fragile veins. Blood sampling is prevalent in general health practice and pathology testing of sampled blood can provide important information e.g. indication of a disease state, cell counts and antibody reactions to name a few. Although blood sampling may be conducted through other known venipuncture techniques, the inventive device and method provides a desirable alternative in cases where traditional venipuncture fails.

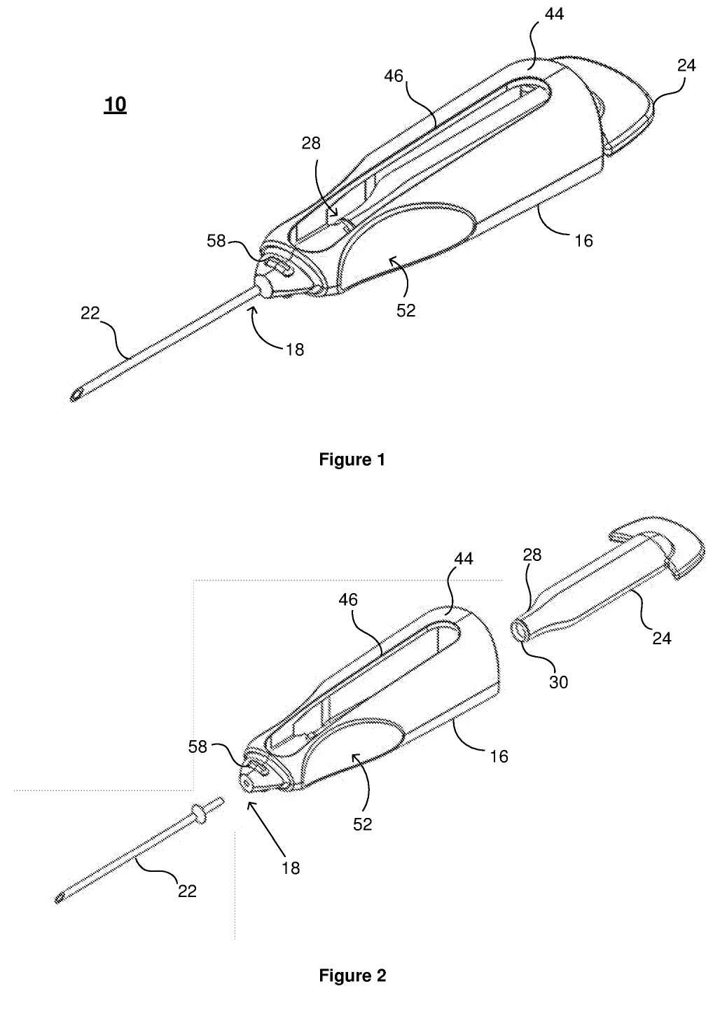

FIG. 1 shows an isometric view of a device 10 for sampling fluid from a subject according to a preferred embodiment of the invention. The device 10 includes a needle 22 and a body 16 couplable with a collecting receptacle 24. The components of the device 10 are shown in an exploded view in FIG. 2.

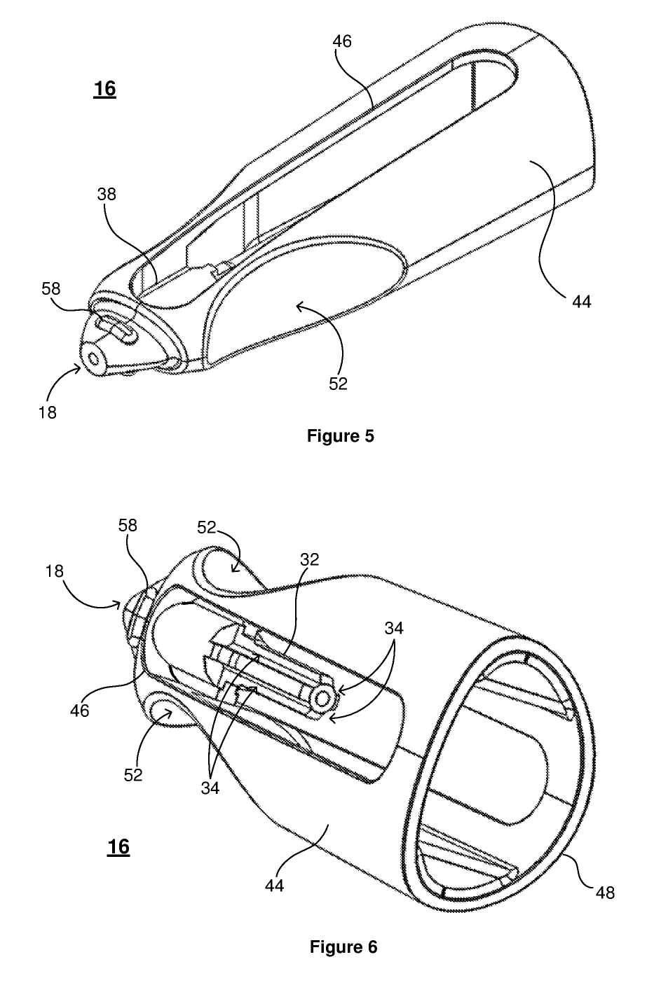

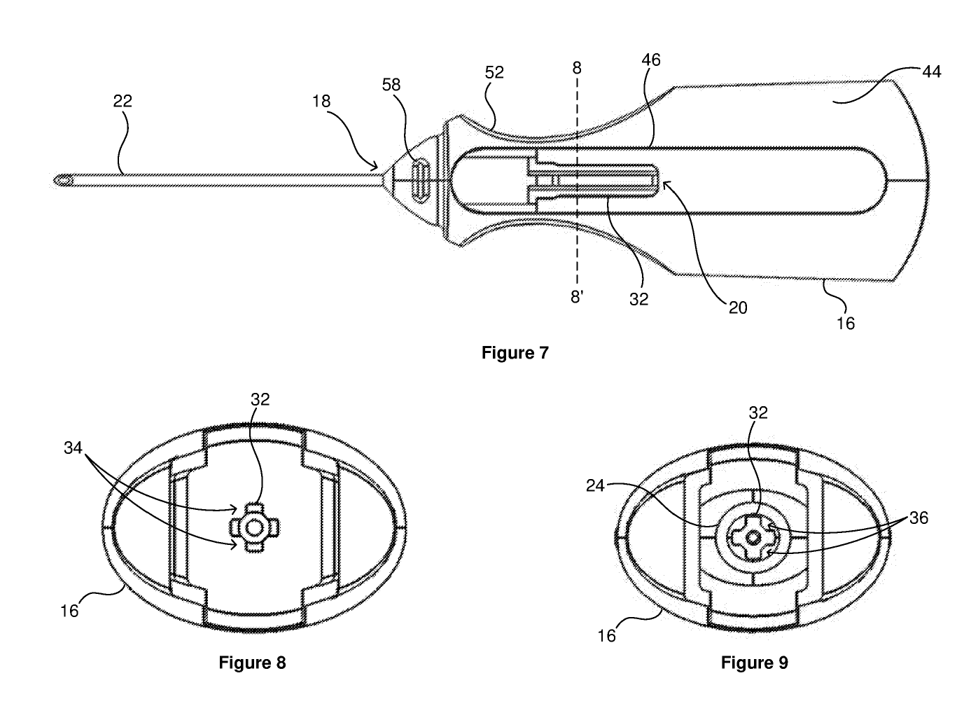

The device 10 includes a body 16 having a fluid inflow end 18 and a fluid outflow end 20 (see also FIGS. 5 and 6). The device 10 further includes a needle 22 which is adapted to penetrate the subject to sample fluid. The needle 22 is in fluid communication with the body 16 at the fluid inflow end 18. The body 16 has a body portion at the fluid outflow end 20 that is couplable with a collecting receptacle 24. The body portion that is couplable with the collecting receptacle 24 is configured such that an interior 26 of the collecting receptacle 24 remains in fluid communication with the ambient environment. This advantageously permits fluid to be passively collected into the collecting receptacle 24 during fluid sampling.

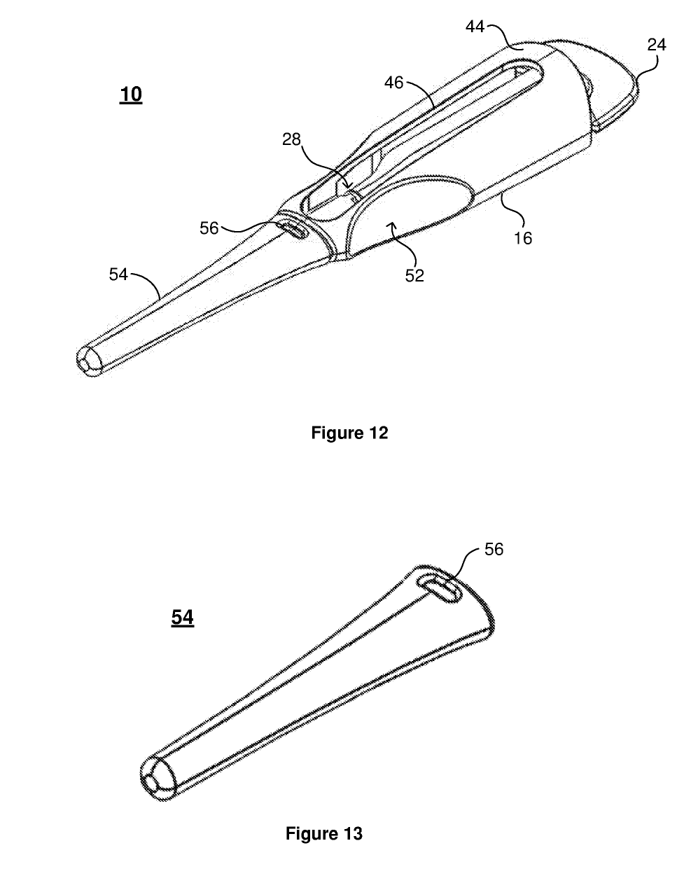

The body portion may be couplable with the collecting receptacle 24 at an end 28 of the collecting receptacle 24 as shown in FIGS. 2 and 11. The collecting receptacle 24 may include at least one opening 30 at end 28 such that the interior 26 of the collecting receptacle 24 remains in fluid communication with the ambient environment. When the device 10 is in use, fluid flows from the subject, through the needle 22 and enters the body 16 via the fluid inflow end 18. The fluid then flows to the fluid outflow end 20 of the body 16 and is collected into the coupled collecting receptacle 24. As well as receiving collected fluid from the fluid outflow end 20 of the body 16, the opening 30 provides a route for air to escape from the interior 26 of the collecting receptacle 24 during fluid collection. Displacement of air from the collecting receptacle 24 is desirable to enable flow of collected/sampled fluid into the collecting receptacle 24 during fluid collection. This allows fluid flowing from the subject to be passively collected into the collecting receptacle 24.

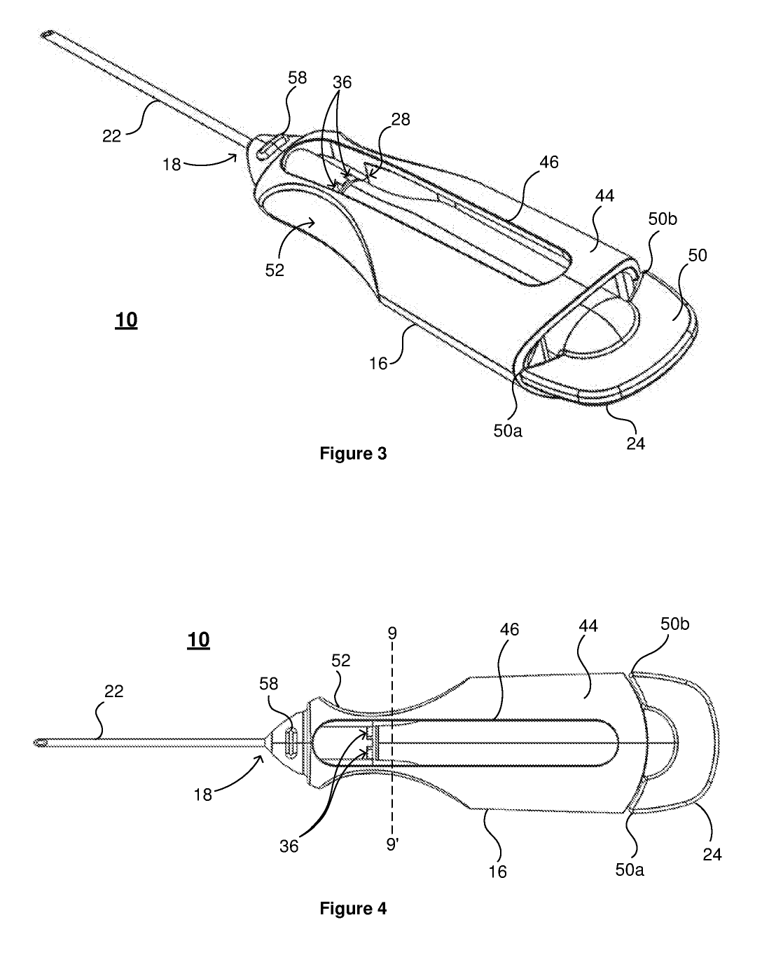

The body portion may be couplable with the collecting receptacle 24 such that it does not entirely occlude the opening 30 of the collecting receptacle 24. When the body portion and the collecting receptacle 24 are coupled together, the opening 30 remains at least partly open to the ambient environment. In some embodiments, the opening 30 is configured so that one or more channels or gaps 36 extend between the body 16 and the interior 26 of the coupled collecting receptacle 24. FIGS. 3 and 4 illustrate such an arrangement and show a rotated and top view, respectively, of the device 10 of FIG. 1. FIGS. 3 and 4 show two gaps 36 in an upper portion of the device 10 that are located between the coupled collecting receptacle 24 and the body 16. Although not shown, there are also two corresponding gaps 36 located in a lower portion of the device 10. The embodiment illustrated provides four gaps 36 for air flow out of the collecting receptacle 24 although fewer or more gaps/channels 36 could be provided. The gaps 36 allow fluid communication between the interior 26 of the coupled collecting receptacle 24 and the ambient environment. Accordingly, air is able to escape from the collecting receptacle 24 through the gaps 36 to enable fluid flow into the collecting receptacle 24 during fluid collection.

Ideally, channels/gaps 36 between the body 16 and the coupled collecting receptacle 24 are formed due to the coupling arrangement between the components. To illustrate this, FIGS. 5 and 6 show enlarged and rotated views, respectively, of the body 16 shown in FIG. 1. FIG. 6 shows that the body portion includes an elongate structure 32 for coupling with the collecting receptacle 24. The elongate structure 32 may be located at the fluid outflow end 20 of the body 16 and include four longitudinal grooves 34 that extend between the fluid inflow end 18 and the fluid outflow end 20. When the elongate structure 32 is coupled with the collecting receptacle 24, each longitudinally extending groove 34 may form a gap between the elongate structure 32 and the coupled collecting receptacle 24 for fluid communication. Alternatively, the arrangement may be reversed wherein grooves or notches are provided along the opening 30 of the collecting receptacle 24 which, when coupled with the elongate structure 32 (with or without longitudinal grooves 36) provide vents or openings through which air in the collecting receptacle 24 may escape during filling with sampled fluid.

The collecting receptacle 24 is couplable with the body 16 through an interference fit. Coupling the components involves sliding the collecting receptacle 24 and the elongate structure 32 together such that the end 28 having the opening 30 faces the fluid outflow end 20 of the body 16 (see also FIGS. 3 and 4). The elongate structure 32 having the smaller external dimension is press-fit into the opening 30 of the collecting receptacle 24. The interface between the end 28 of the coupled collecting receptacle 24 and the body 16 is such that the components are not easily separable. In alternative embodiments, the interference fit could be replaced by e.g. a snap-fit engagement having snap features positioned between the body 16 and coupled collecting receptacle 24 (not shown).

The interface between the end 28 of the coupled collecting receptacle 24 and the body 16 is unsealed due to the gaps 36 as shown in FIGS. 3 and 4. The gaps 36 result from a mismatch in cross-sectional shape that occurs when the elongate structure 32 and collecting receptacle 24 are coupled together. The mismatch is more clearly illustrated in FIGS. 7 to 9. FIG. 7 depicts a top view of the device 10 of FIG. 1 that omits the collecting receptacle 24 to show the elongate structure 32. FIG. 8 shows a sectional view of the elongate structure 32 along the line 8-8′ in FIG. 7 viewed in the direction away from the fluid inflow end 18 of the body 16. The four longitudinal grooves 34 that extend along the elongate structure 32 provide a generally star or cross-shaped cross-section (see also FIG. 6).

In contrast, the cross-sectional shape of the end 28 of the coupled collecting receptacle 24 is generally circular. This is illustrated by the cross-section in FIG. 9 which shows a sectional view along the line 9-9′ in FIG. 4 viewed in the direction towards the fluid inflow end 18 of the body 16. FIG. 9 shows the star or cross-shaped cross-section of the elongate structure 34 positioned inside the circular cross-section of the collecting receptacle 24. The mismatch between the cross-sections provides channels/gaps 36 between the periphery of the elongate structure 32 and opening 30 of the collecting receptacle 24 enabling air to be displaced as the collecting receptacle 24 fills with fluid during fluid collection.

Since the channels 36 are positioned within the coupled collecting receptacle 24, they may become filled with sampled fluid during fluid collection and become blocked. If all the channels 36 are blocked, the opening 30 of the collecting receptacle 24 will no longer allow fluid communication with the ambient environment.

To address this, the grooves 34 of the elongate structure 32 may be arranged to mitigate simultaneous occlusion of gaps 36 between the elongate structure 32 and the coupled collecting receptacle 24 with sampled fluid from the subject during fluid collection. The longitudinal grooves 34 may be spaced apart circumferentially around the elongate structure 32 to enable formation of correspondingly arranged channels 36 between the elongate structure 32 and the collecting receptacle 24 when coupled. The spacing of the channels 36 reduces the likelihood of all channels 36 becoming blocked during fluid collection. Depending on the orientation of the device 10, the channels 36 which are positioned in a lower portion of the device 10 may become blocked due to pooling of the sampled fluid under gravity. However, the channels in an upper portion of the device 10 are more elevated and therefore likely to remain free of the sampled fluid as it is collected in the collecting receptacle 24.

In some embodiments, the body 16 may be releasably couplable with the collecting receptacle 24. One or both of the body 16 and collecting receptacle 24 may include an engagement surface that allows the collecting receptacle 24 to be controllably decoupled e.g. when rotated with respect to the body 16. Ideally, the engagement surface urges the collecting receptacle 24 and body 16 apart when there is relative rotational movement between the components, thereby decoupling the collecting receptacle 24 and the body 16.

Preferably, there are engagement surfaces on the body 16 and the collecting receptacle 24 to provide controlled active separation during decoupling. As particularly shown in FIGS. 3 and 6, the engagement surface of the body 16 is a contoured edge 48 along a rear portion thereof. FIG. 3 shows that the engagement surface of the collecting receptacle 24 is a contoured edge on a portion 50 thereof. The corresponding contoured edges of the body 16 and the collecting receptacle 24 may be configured to rest in abutment when the body 16 and collecting receptacle 24 are coupled. The corresponding contoured edges may also be configured to urge apart the body 16 and the collecting receptacle 24 when one is rotated relative to the other. It is the relative movement between the abutting contoured edges that urges the components apart.

The contoured edge of the collecting receptacle 24 may be flanked by winged/flange portions 50a and 50b each having an edge that is shaped to abut against the contoured edge 48 of the body 16 when the body 16 and the collecting receptacle 24 are coupled for fluid collection as shown in FIGS. 2 and 11. When the collecting receptacle 24 is rotated with respect to the body 16 (or vice versa), the edge of the winged portions 50a, 50b travel along the contoured edge 48 of the body 16 and are gently urged away from the body 16. Advantageously, controlled separation of the body 16 and the collecting receptacle 24 reduces the risk of spillage of the collected fluid after fluid sampling.

The collecting receptacle 24 may be rotated with respect to the body 16 by an operator grasping and rotating the winged portions 50a, 50b. The rotation may be clockwise or anticlockwise with respect to the body 16. Further, the edge of the winged portions 50a, 50b that engages the contoured edge 48 of the body 16 may also be contoured. As shown in FIGS. 3 and 6, the portion 50 having winged portions 50a, 50b is located to one end of the collecting receptacle 24, which advantageously is the end to which force is applied to rotate the collecting receptacle 24. This enables less force to be applied by an operator and provides for easier separation of the components. However, this need not be the case and the collecting receptacle 24 may be include an engagement surface to allow decoupling when rotated with respect to the body 16 along any part of the receptacle 24. Similarly, the embodiment illustrated in FIGS. 3 and 6 shows a frame 44 of the body 16 that includes the contoured edge 48. However, the engagement surface of the body 16 may be along any part of the body 16, such as on the elongate structure 32, to provide the controlled separation from the collecting receptacle 24.

In alternative embodiments, the body 16 may be releasably couplable with the collecting receptacle 24 through other means. For example, the body 16 and collecting receptacle 24 may be threaded for threaded engagement of the components. Alternatively, the body 16 and collecting receptacle 24 may releasably engage through friction-fit (such as a press-fit) between the components having different coupling diameters. Alternatively, the body 16 and collecting receptacle 24 may be releasably couplable through a bayonet fitting or the like.

In some embodiments, the body 16 of the device 10 includes a transparent portion 38 to enable observation of fluid flash-back into the body 16 when in use. This assists in confirming good location of the needle 22 for fluid access at an anatomical feature of the subject, such as a blood vessel, cyst, abscess or blister to name a few. In the case of venous blood sampling, the operator can confirm that the needle 22 is correctly positioned in a vein of the subject when blood flash-back is visible through the transparent portion 38 of the body 16 of the device 10.

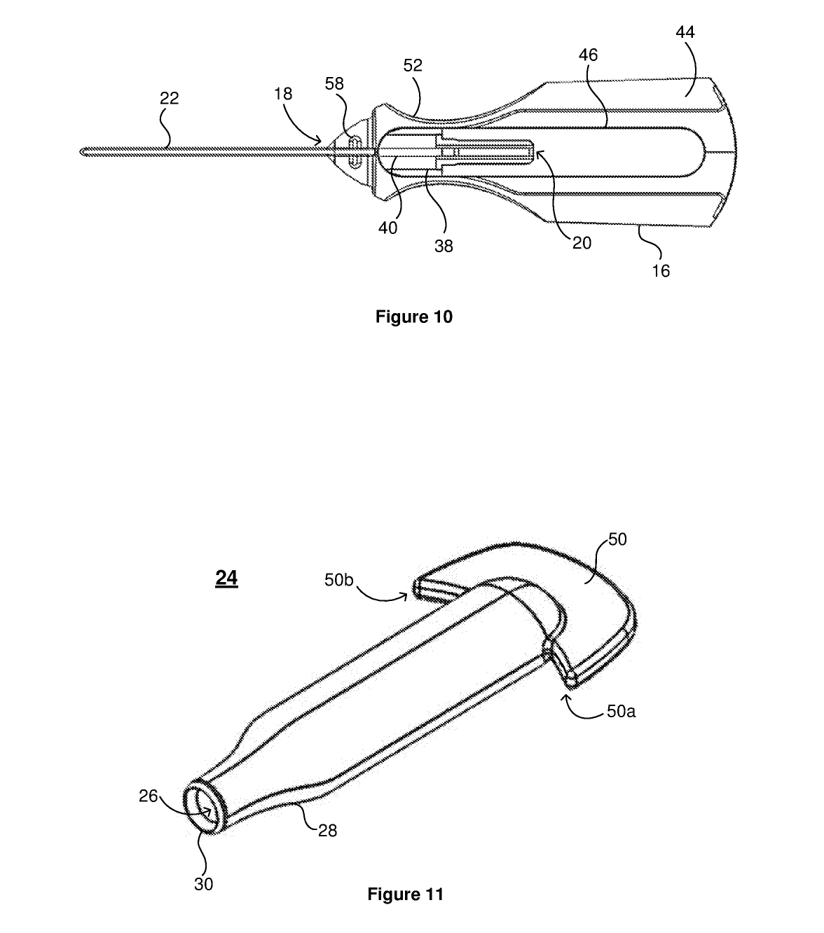

FIG. 10 shows a top view of the device 10 from FIG. 1 that omits the collecting receptacle and shows the transparent portion 38 as a chamber or window. The transparent portion 38 is positioned between the fluid inflow end 18 and the fluid outflow end 20 and shows the elongate structure 32 inside the device body 16. In this embodiment, the needle 22 and body 16 are both transparent for inspecting the flow path of the sampled fluid through the device 10. However, the needle 22 need not be transparent and the body 16 may be transparent only at the transparent portion 38. During fluid sampling using the embodiment shown, the sampled fluid will flow through the needle, enter the body 16 at the fluid inflow end 18, fill the transparent portion 38 and exit the body 16 at the fluid outflow end 20 for collection in the collecting receptacle 24 (not shown). Upon inserting the needle 22, fluid flash-back will be visible at the fluid inflow end 18 through the transparent portion 38 if the needle is inserted correctly.

Preferably, the body 16 and collecting receptacle 24 (not shown) are transparent so that flow of the sampled fluid through the body 16 may be visible to an operator when using the device 10. The entire device 10 or one or more of the needle 22, body 16 and collecting receptacle 24 may be manufactured from a transparent material. The transparent material is ideally the kind used for syringes and tubes often used in medical applications. Additionally/alternatively, the transparent portion 38 may be shaped to form a lens that magnifies viewing of the fluid flash-back (not shown). The lens magnification may be advantageous where the dimensions of the device 10 are restricted due to ergonomic requirements or where the amount of fluid flash-back is minimal, such as for venous blood sampling with a subject having small or fragile veins.

In other embodiments, the body 16 comprises a lumen 40 between the fluid inflow end 18 and the fluid outflow end 20 as shown in FIG. 10. The lumen 40 guides the sampled fluid such that it is directed towards the fluid outflow end 20 to facilitate collection in a collecting receptacle 24 coupled to the body 16. The lumen 40 encourages fluid to flow through the body 16 along a direct pathway, rather than “flooding” the interior surface of the body 16. The transparent portion 38 may make visible the lumen 40 as shown in FIG. 10 such that fluid flash-back into the lumen 40 at the fluid inflow end 18 can be observed when the device 10 is in use.

The device 10 may be configured to self-anchor in the subject during fluid sampling and collection into the collecting receptacle 24. The relative lengths of the needle 22 and the body 16 with coupled collecting receptacle 24 assists with self-anchoring of the device 10. That is, the length of the needle 22 relative to the size and weight of the body 16 with coupled collecting receptacle 24 is such that there is self-anchoring during use while the subject is restrained. When the size of the body 16 with coupled collecting receptacle 24 is increased relative to the needle length, the effects of gravity and the greater bulk and mass give rise to a pull-out force which exceeds the ability of the device 10 to self-anchor.

To achieve self-anchoring, typically the needle has a length of approximately 10 to 40 mm and the length is selected to be suitable for the fluid collection application. It may be preferred for paediatric applications that the needle length is generally in the range of 10 to 20 mm, and more preferably, the needle length is about 14 mm. The body 16 with the coupled collecting receptacle 24 generally has a length of 40 to 80 mm, and more preferably, 50 to 60 mm. The width and depth of the body 16 with coupled collecting receptacle 24 may generally be 20 mm and 10 mm, respectively. However, the relative dimensions of the needle 22 and body 16 with coupled collecting receptacle 24 may be varied depending on the fluid collection application but ideally facilitate self-anchoring of the device 10 into the subject during fluid sampling.

The relative lengths of the needle 22 and body 16 with coupled collecting receptacle 24 are considerably smaller than other devices used in the art, such as those described for venipuncture. This beneficially minimises deadspace in the device 10 and the risk of clotting when the fluid sampled is blood.

When sampling venous blood from paediatric subjects and other subjects with small and/or fragile veins, the needle 22 is typically of size 21 or 23 gauge. Ideally, the needle 22 should be no smaller than 25 gauge. This may seem counterintuitive for blood sampling from smaller veins, however the inventors have determined that a needle size smaller than 25 gauge limits blood flow to an extent that passive fluid collection under venous pressure alone may be inhibited.

In FIGS. 1, 3, 4, 7 and 10, the fluid inflow end 18 of the body 16 is shown directly coupled to the needle 22. The direct coupling may be through a permanent connection and/or involve bonding and sealing of the needle 22 to the body 16 using adhesive, which may involve methods as known in the art. The adhesive approach has the advantage of eliminating any gap between the needle 22 and the body 16, which is likely to be present due to the differing part tolerances. In other embodiments, the device 10 may include a releasable coupling (not shown) between the body 16 and needle 22. This feature allows the device 10 to be adapted for different sized needles, enabling its use in a wider range of subjects. The body 16 may be releasably couplable with the needle 22 by a threaded engagement, e.g. comprising an external thread located on the needle 22, and an internal thread located in the wall of the body 16. The threads are adapted to lock effectively during fluid sampling. Alternatively, the needle 22 may be press-fit into the body 16 using an interference fit or using other coupling means.

FIG. 11 provides an enlarged view of the collecting receptacle 24 that is releasably couplable with the device 10 as shown in FIG. 1 for sampling fluid 12 from a subject. The collecting receptacle 24 is adapted to maintain an interior 26 of the collecting receptacle 24 in fluid communication with the ambient environment while coupled to the fluid sampling device 10. This advantageously permits fluid to be passively collected into the collecting receptacle 24 during fluid sampling.

The device 10 and collecting receptacle 24 are releasably couplable at end 28 of the collecting receptacle 24. The end 28 includes an opening 30 for receiving sampled fluid into the interior 26 of the collecting receptacle 24 during fluid collection. The collecting receptacle 24 may be couplable with the device 10 such that the opening 30 is not fully occluded by the coupling so as to maintain fluid communication between the interior 26 of the collecting receptacle 24 and the ambient environment.

In some embodiments, one or both of the device 10 and collecting receptacle 24 include an engagement surface that urges the collecting receptacle 24 and device 10 apart when there is relative rotational movement between the components. This enables decoupling of the collecting receptacle 24 and the device 10. The engagement surface may include a contoured edge on a portion 50 of the collecting receptacle 24 that is configured to abut against a contoured edge 48 of the device 10 when coupled thereto, as previously described. When one of the collecting receptacle 24 and the device 10 is rotated relative to the other of the device 10 and the collecting receptacle 24, the relative movement between their abutting portions urges the components apart. The abutting portion of the collecting receptacle 24 may include winged portions 50a, 50b having an edge that is shaped to abut against the contoured edge 48 of the body 16. The collecting receptacle 24 may be rotatable by an operator grasping and rotating the winged portions 50a, 50b.

In some embodiments, at least part of the walls of the collecting receptacle 24 are compliant to allow sampled fluid to be expelled therefrom after fluid sampling by squeezing or compressing. The compliance may be achieved through the walls of the collecting receptacle including a material that flexes or compresses when force is applied. Alternatively, the walls may be shaped to achieve compliant parts that enable sampled fluid to be expelled therefrom such as through tapering of the width and/or thickness. The body 16 of the device 10 may include a protective frame 44 as shown in FIGS. 1 to 7 and 10. The protective frame 44 at least partly surrounds at least the compliant parts of the collecting receptacle 24 when coupled to the body 16. Thus, the frame 44 protects the collecting receptacle 24 from inadvertent squeezing or compressing by an operator of the device 10 during fluid sampling.

The collecting receptacle 24 may also include an indicator (not shown) to allow the amount of fluid collected to be estimated or measured during fluid collection. This may be useful in embodiments where the body 16 is at least partly transparent so that the fluid level may be observed during collection. The indicator may comprise a transparent portion in the collecting receptacle 24 that enables an operator to view the fluid level or level of fullness inside collecting receptacle 24. In some embodiments, the frame 44 includes an open or transparent slot 46 as shown in FIGS. 1 to 7 and 10 through which the fluid level in the collecting receptacle 24 can be observed during fluid collection. The slot 46 is particularly useful in embodiments where the collecting receptacle 24 is transparent since it allows viewing of the fluid level through the slot 46. Markers may be included on one or both of the body 16 and the collecting receptacle 24 to indicate the volume of fluid collected (not shown). The markers may indicate actual volumes, e.g. 10 μL, 20 μL, 30 μL etc., or relative volumes e.g. 25%, 50% and 75% of the capacity of the collecting receptacle 24.

In some embodiments, the collecting receptacle 24 has a collection capacity in the range of 0.3 mL to 20 mL. In paediatric applications, it may be preferable that the collecting receptacle 24 has a lesser volume capacity in the range of 0.3 mL to 2 mL, and more preferably a volume capacity of about 1 mL. Alternatively, in other applications the volume capacity may be e.g. 2 mL to 10 mL, and more preferably, 3 to 5 mL. However, the volume capacity may be 10 mL to 20 mL in applications requiring larger volumes, such as in veterinary applications. It will be appreciated that the volume capacity of the collecting receptacle 24 may exceed the range of 0.3 mL to 20 mL, depending on the fluid sampling application, as would be understood by a person skilled in the art. The volume capacity of the collecting receptacle 24 may be increased or decreased as required by selecting collecting receptacles 24 of a volume capacity suitable for the particular fluid sampling requirements.

In some embodiments, the device 10 may include a safety mechanism 54 to prevent inadvertent needle penetration (i.e. needle stick injury) before or after fluid sampling. FIG. 12 shows an isometric view of a modified device 10 which includes a cap 54 applied over the needle 22 and coupled with the body 16 at the fluid inflow end 18. The cap 54 is configured for snap-fit engagement with the body 16. The cap 54 is couplable with the body 16 through a snap interface that includes slots 56 on the cap 54 which are sized for engaging corresponding detents 58 on the body 16 at the fluid inflow end 18 as shown in FIGS. 12 and 13. Alternatively, the snap interface may be reversed so the detents 58 are positioned on the cap 54 and the slots 56 are found on the body 16 at the fluid inflow end 18. The device 10 includes two pairs of slots/detents 56, 58 on a top and bottom surface of the device 10 for secure engagement. However, there need only be a single slot/detent 56, 58 to provide the snap-fit engagement.

The cap 54 may couple with the body 16 through alternate arrangements to snap-fit engagement (not shown). For example, the cap 54 may be couplable with the body 16 through an interference fit where one component press-fits into the other. Preferably, the body 16 has a smaller external dimension such that it press-fits into the cap 54. However, this may be reversed such that the cap 54 has the smaller external dimension and press-fits into the body 16. To uncouple the components, one or both of the cap 54 and body 16 may have an engagement surface that allows decoupling when the cap 54 is rotated with respect to the body 16 (not shown). For example, the body 16 may include a contoured edge such that when the cap 54 is rotated with respect to the body 16, the cap 54 travels along the contoured edge and is pushed away from the body 16. Additionally/alternatively, the cap 54 may couple with the body 16 through threaded engagement or a bayonet fitting.

Other safety mechanisms for preventing inadvertent needle penetration may be known in the art and may include a cork or stopper into which the needle tip is inserted before or after fluid sampling. In other embodiments, the safety mechanism may include the feature of the needle 22 being retractable into the body 16 where the respective dimensions permit.

In some embodiments, the body 16 may include contoured surfaces 52 as shown in FIGS. 1 to 7, 10 and 12 that are adapted to be held by an operator while manipulating the device 10. The contoured surfaces 52 may be texturized for improved gripping by the operator and/or may be rubberised to provide gripping/grasping contact surfaces (not shown). The contoured surfaces 52 allow an insertion force to be applied to the needle 22 by the operator such that the needle 22 can be more easily inserted into an anatomical feature of the subject for fluid sampling. The contoured surfaces 52 are shown in the respective figures on opposing sides of the body 16 to assist the operator in positioning the device 10 in the correction orientation during fluid sampling (i.e. having slot 46 viewable from a top of the device 10). The figures also show the contoured surfaces 52 shaped to curve inwards towards an interior of the body 16 to provide grooves for finger gripping by an operator. However, the contoured surfaces 52 may alternatively protrude outwards from the body 16 to provide gripping/grasping surfaces or sit flush with the body 16 and include texturized gripping/grasping surfaces (not shown).

Typically, the collecting receptacle 24 is designed for single use with the device 10 for collecting fluid sampled from the subject. In some embodiments, the collecting receptacle 24 remains coupled to the body 16 of the device 10 once filled and until such a time that the collected fluid is analysed or tested, such as in a pathology laboratory. This ensures that the collected fluid in the collecting receptacle 24 remains isolated from the surrounding environment during transport or movement of the device 10 so as to minimise the likelihood of spillage or leakage of the sampled fluid. This is particularly important when the sampled fluid is blood, so that the device 10 and method for using the device 10 for blood collection meets universal safety precautions for sampling blood in a sanitary and clean as possible manner.

In other embodiments, the device 10 having the needle 22 and body 16 may be used many times for collection of sampled fluid from the same or different subjects assuming sterilisation occurs between subjects. The collecting receptacle 24 is ideally replaced each time the device 10 is used for taking a fluid sample from a subject. Each collecting receptacle 24 may have a closure applied once filled, or may be emptied (e.g. by squeezing the collected fluid out through opening 30) into a vial for use in pathology testing or the like. In embodiments where a closure is applied to the collecting receptacle 24, such as a cap or seal, it is desirable that the closure is applied such that the collected fluid in the collecting receptacle 24 remains isolated from the surrounding environment. This ensures that the likelihood of spillage or leakage of the sampled fluid is minimised during transport of the collecting receptacle 24, e.g. to a pathology laboratory. Again, this is particularly important when the sampled fluid is blood in order to meet universal safety precautions for sampling blood in a sanitary and clean as possible manner. In embodiments where the collected fluid is expelled into a vial or separate container, it is desirable that the sampled fluid is dripped from opening 30 (e.g. passively under the influence of gravity or by squeezing a compliant collecting receptacle 24) to the vial or separate container. Ideally, the transfer of sampled fluid is performed in a contained and sanitary manner so as to avoid the likelihood of leakage or spillage, and ideally meet universal safety precautions for sampling blood in a sanitary and clean as possible manner.

Preferably, the body 16 of the device 10 is manufactured by injection moulding and the collecting receptacle 24 is manufactured by blow moulding to form the hollow interior 26. Additionally/alternatively, the collecting receptacle 24 can be made in two parts and welded, glued or bonded together to form the hollow interior 26. The relative sizes of the body 16 and collecting receptacle 24 can be adjusted depending on the volume requirements for the required fluid sampling.

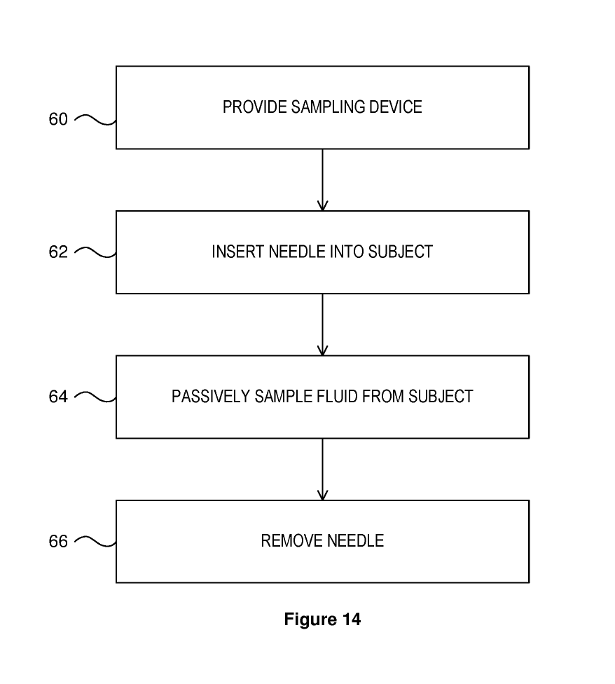

FIG. 14 illustrates a flow chart showing steps in a method for sampling fluid from a subject according to an embodiment of the invention. The method includes at step 60 providing a sampling device 10 including a body 16 having a fluid inflow end 18 and a needle 22 at a fluid outflow end 20 of the body 16. The needle 22 is inserted into the subject at step 62. The method includes sampling fluid at step 64 by allowing fluid to flow from the subject, through the needle 22, and into the body 16 through the fluid inflow end 18 for collection at the fluid outflow end 20 in a collecting receptacle 24. The needle 22 is removed from the subject at step 66. The method enables fluid to be passively collected into the collecting receptacle 24 during fluid sampling through an arrangement between the fluid outflow end 20 of the body 16 and collecting receptacle 24 that maintains an interior 26 of the collecting receptacle 24 in fluid communication with the ambient environment.

The step 62 of inserting the needle 22 may include an operator applying force to the body 16 using their finger grip on contoured surfaces 52 of the body 16 to gently drive the needle 22 into the subject at an appropriate anatomical feature. The anatomical feature of the subject may include a blood vessel, cyst, abscess or blister to name a few. In some embodiments, the needle 22 penetrates the subject to a needle depth sufficient to enable the device 10 to self-anchor and remains in the subject during fluid sampling and collection. After anchoring is achieved, the method may include the step of the operator releasing the contoured surfaces 52 of the body 16. Fluid is then sampled at step 64 by allowing it to flow from the subject, through the needle 22, and into the body 16 through the fluid inflow end 18 for collection in the collecting receptacle 24 at the fluid outflow end 20. The fluid may flow or drip into the collecting receptacle 24. In some embodiments, the body 16 may be taped onto the subject using medical tape to further anchor the device 10 during fluid sampling.

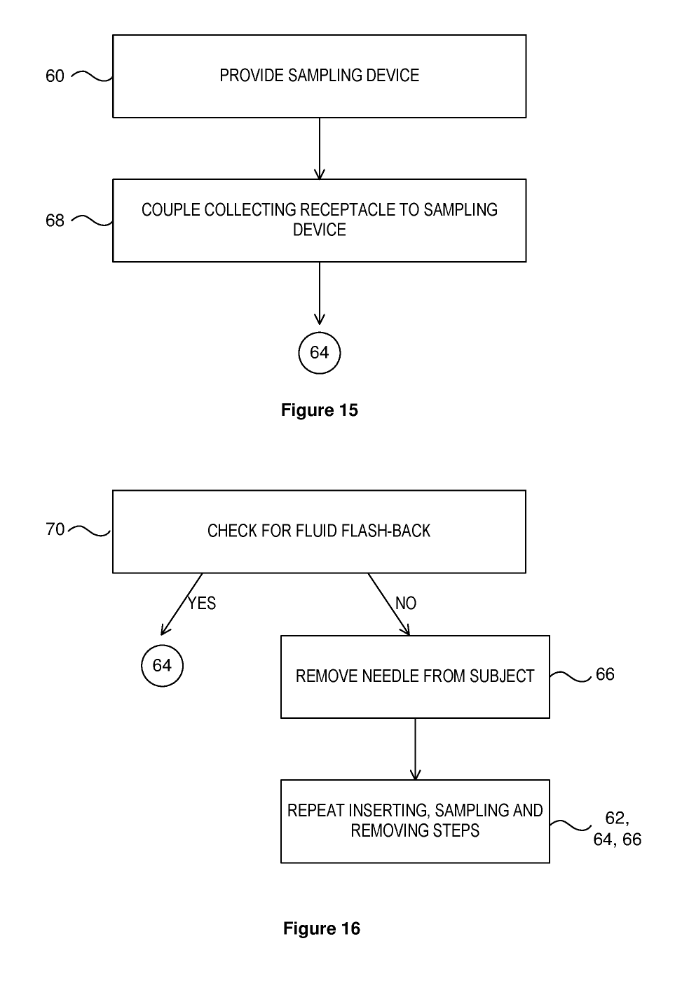

FIG. 15 illustrates a flow chart showing further steps in the method shown in FIG. 14. In some embodiments, after providing the sampling device 10 at step 60, the method includes the step 68 of coupling the collecting receptacle 24 to the body 16 at the fluid outflow end 20. The coupling is such that the interior 26 of the collecting receptacle 24 is in fluid communication with the ambient environment. Following coupling of the collecting receptacle 24, the method includes sampling fluid at step 64 as shown in FIG. 14.

In some embodiments, the method includes at step 70 checking for fluid flash-back through at least a transparent portion 38 of the body 16. Advantageously, the contoured surfaces 52 of the body 16 are able to be grasped by the operator that allows the transparent portion 38 of the device 10 to be unobscured from the operator's view so that fluid flash-back can be observed. FIG. 16 illustrates a flow chart including further steps in the method shown in FIGS. 14 and 15 relating to checking for fluid flash-back. The appearance of flash-back visible through the transparent portion 38 indicates that the needle 22 is correctly positioned. If fluid flash-back is present, the method includes the step 64 of sampling fluid. However, if fluid flash-back is not present, the method includes the step 66 of removing the needle from the subject and making another attempt at positioning by repeating the inserting 62, sampling 64 and removing 66 steps.

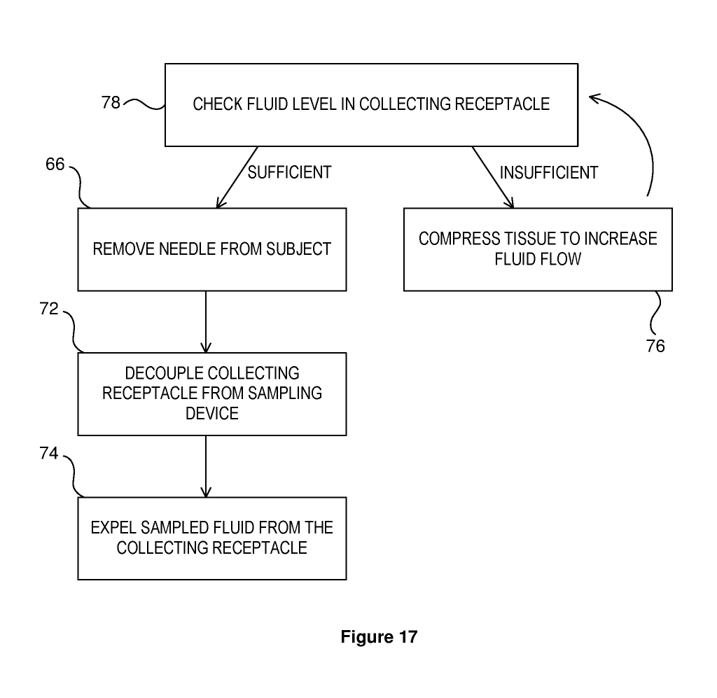

The method may also include further steps relating to checking the fluid level as illustrated in the flow chart of FIG. 17. In some embodiments, the method includes at step 78 checking the fluid level in the collecting receptacle 24 during fluid sampling by inspecting an indicator on the collecting receptacle 24. The indicator may be the visible level of the fluid in the collecting receptacle 24 or one or more indicator lines marked on the receptacle 24. If the fluid level indicator suggests that sufficient fluid has been collected, the method includes the step 66 of removing the needle 22 from the subject. The method may further include the step 72 of decoupling the collecting receptacle 24 from the sampling device 10 by rotating it with respect to the body 16. In embodiments where the collecting receptacle 24 is made of compliant material, the method may include the step 74 of squeezing or compressing the decoupled collecting receptacle 24 to allow the sampled fluid to be expelled from the collecting receptacle 24 into a device, collection vessel or pathology tube. An operator may desire to aliquot the sampled fluid into separate vials or containers for various forms of pathology testing. Additionally/alternatively, the sampled fluid may be expelled into specific vials or containers that are used with equipment for pathology testing.

In some embodiments, the method may include the step 68 of coupling another collecting receptacle 24 to the body 16 prior to the step 66 of removing the needle from the subject. In this way, fluid may be collected from the subject and fill more than one collecting receptacle 24. This advantageously means that the operator does not need to aliquot the sampled fluid into separate vials or containers for various forms of pathology testing. The method may also include applying a cap to the opening 30 of the collecting receptacles 24 once filled and decoupled from the body 16.

Advantageously, using the inventive device 10 and method for blood sampling requires only venous pressure for fluid to flow from the subject, through the needle 22, and into the body 16 for collection in collecting receptacle 24. The blood is sampled without the use of suction which can cause small and fragile veins to collapse. If the amount of fluid collected is insufficient, or is slow or difficult to collect under venous pressure alone, the method may include the step 76 of compressing tissue of the subject near the needle 22 with the application of pressure increasing fluid flow into the needle 22. This is illustrated in the flow chart of FIG. 17 and may involve the operator compressing and releasing (e.g. palpating) the tissue of the subject near the needle 22 with the operator's hand. Following this step, the fluid level in the collecting receptacle 24 may again be checked at step 78.

In some embodiments, the sampled fluid in the collecting receptacle 24 has a volume in the range of 0.3 mL to 20 mL. In paediatric applications, preferably the sampled fluid has a volume in the range of 0.3 mL to 2 mL, and more preferably a volume of about 1 mL. Alternatively, in other applications the volume is in the range of 2 mL to 10 mL, and more preferably, 3 to 5 mL. However, the volume may be in the range of 10 mL to 20 mL for applications requiring large volumes, such as required in veterinary applications. The volume may be increased or decreased as required by selecting a collecting receptacle 24 ideal for the particular fluid sampling required from the subject. It will be appreciated that the sampled fluid may have a volume exceeding the range of 0.3 mL to 20 mL, depending on the fluid sampling application, as would be understood by a person skilled in the art.

The sampling device 10 and collecting receptacle 24 may be provided in kit form (not shown). In a preferred embodiment of the invention, a kit includes a sampling device 10 and one or more collecting receptacles 24. The sampling device 10 includes a body 16 having a fluid inflow end 18 and a fluid outflow end 20, and a needle 22 at the fluid inflow end 18 that is adapted to penetrate a subject to sample fluid. The body 16 also includes a body portion at the fluid outflow end 20 that is couplable with the one or more collecting receptacles 24. The body portion is configured such that an interior 26 of the one or more collecting receptacles 24 remains in fluid communication with the ambient environment to permit fluid to be passively collected into the one or more collecting receptacles 24 during fluid sampling.

In some embodiments, the volume capacity of the collecting receptacles 24 is selected from one of: in the range of 0.3 mL to 20 mL, in the range of 0.3 mL to 2 mL and about 1 mL depending on the application. The volume capacity may be preferably in the range of 3 to 5 mL. It will be appreciated that the volume capacity may exceed the range of 0.3 mL to 20 mL, depending on the fluid sampling application, as would be understood by a person skilled in the art. The kit may include two or more collecting receptacles 24 each having a different volume capacity. This advantageously allows an operator of the sampling device 10 to select collecting receptacles 24 with a volume capacity suitable for the fluid sampling application.

In some embodiments, the collecting receptacles 24 include an anticoagulant. The anticoagulant may be selected from one of the group including: sodium heparin, potassium oxalate, ethylene diamine tetraacetic acid (EDTA), sodium citrate, acid citrate dextrose (ACD), sodium polyanethol sulfonate (SPS) and thrombin. Preferably, the kit includes two or more collecting receptacles 24 each having a different anticoagulant. This advantageously allows an operator of the sampling device 10 to select collecting receptacles 24 with an anticoagulant suitable for the fluid sampling application.

In a preferred embodiment of the invention, there is a method for sampling fluid from a subject using the kit as described herein. The method may include one or more of the steps as described herein and with reference to the flow charts illustrated in FIGS. 14 to 17.

Applicability of the inventive device, method and kit is not limited to the sampling of blood or fluid from subjects with small and/or fragile veins. Other candidates may include subjects whom are uncooperative or difficult to immobilise, such as paediatric patients or animals in veterinary applications. Advantageously, the device can be secured during fluid sampling through self-anchoring allowing the operator a free hand to hold a fluid collecting receptacle. This may result in more successful fluid sampling, ensuring that a sample is obtained more consistently on the first attempt. In some embodiments additional anchoring may be obtained by taping the body 16 of the device 10 to the subject near the needle insertion site.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present invention as defined in the claims appended hereto.

It is to be understood that the following claims are provided by way of example only, and are not intended to limit the scope of what may be claimed in any future application. Features may be added to or omitted from the claims at a later date so as to further define or re-define the invention or inventions.

1. A device for sampling fluid from a subject, the device including:
a body having a fluid inflow end and a fluid outflow end; and
a needle adapted to penetrate the subject to sample fluid, wherein the needle is in fluid communication with the body at the fluid inflow end,
wherein the body has a body portion at the fluid outflow end that is couplable with a collecting receptacle, and
wherein the body portion that is couplable with the collecting receptacle is configured such that an interior of the collecting receptacle remains in fluid communication with the ambient environment to permit fluid to be passively collected into the collecting receptacle during fluid sampling.

2. The device according to claim 1, wherein the body portion is couplable with the collecting receptacle such that it does not entirely occlude an opening of the collecting receptacle.

3. The device according to claim 2, wherein the body portion includes an elongate structure for coupling with the collecting receptacle, the elongate structure having one or more longitudinally extending grooves that, when coupled with the collecting receptacle each form a gap between the elongate structure and coupled collecting receptacle for fluid communication.

4. The device according to claim 3, wherein the grooves of the elongate structure are arranged to mitigate simultaneous occlusion of gaps between the elongate structure and the coupled collecting receptacle with sampled fluid from the subject during fluid collection.

5. The device according to claim 4, wherein the one or more longitudinally extending grooves are spaced apart circumferentially around the elongate structure to form correspondingly arranged channels between the elongate structure and the collecting receptacle when coupled.

6. The device according to any one of the preceding claims, wherein the body includes a transparent portion for observing fluid flash-back into the body during fluid sampling.

7. The device according to claim 6, wherein the transparent portion is shaped to form a lens that magnifies viewing of the fluid flash-back.

8. The device according to any one of the preceding claims, wherein the body includes a lumen between the fluid inflow end and the fluid outflow end for guiding the sampled fluid towards the fluid outflow end for collection in the collecting receptacle.

9. The device according to claim 8 when appended to claim 6 or claim 7, wherein the lumen is visible through the transparent portion and fluid flash-back into the lumen at the fluid inflow end is observable during fluid sampling.

10. The device according to any one of the preceding claims, wherein the body is configured to self-anchor the needle in the subject during fluid sampling.

11. The device according to any one of the preceding claims, wherein at least part of the collecting receptacle is compliant to allow the sampled fluid to be expelled therefrom by squeezing or compressing.

12. The device according to claim 11, wherein the body includes a protective frame configured to protect the compliant part of the collecting receptacle when the body and collecting receptacle are coupled together.

13. The device according to claim 12, wherein the frame includes a slot for inspecting the fluid level in the collecting receptacle during fluid collection.

14. The device according to any one of the preceding claims, wherein the collecting receptacle includes an indicator to allow the amount of fluid collected to be ascertained during fluid collection.

15. The device according to any one of the preceding claims, wherein the device is substantially transparent for observing fluid flow through the device during fluid sampling.

16. The device according to any one of the preceding claims, wherein the body is releasably couplable with the collecting receptacle.

17. The device according to claim 16, wherein one or both of the body and collecting receptacle include an engagement surface that urges the collecting receptacle and body apart when there is relative rotational movement between the components, thereby decoupling the collecting receptacle and the body.

18. The device according to claim 17, wherein the engagement surface includes a contoured edge on the body and a corresponding contoured edge on a portion of the collecting receptacle, the corresponding contoured edges being configured to rest in abutment when the body and the collecting receptacle are coupled, and to urge apart the body and the collecting receptacle when one is rotated relative to the other, wherein the relative movement between the abutting contoured edges urges the components apart.

19. The device according to claim 18, wherein the contoured edge of the collecting receptacle includes at least one winged portion having an edge that is shaped to abut against the contoured edge of the body, and wherein the collecting receptacle is rotated relative to the body by an operator grasping and rotating the winged portion.

20. The device according to claim 18 or claim 19 when claim 16 is appended to claim 12 or claim 13, wherein the protective frame includes the contoured edge of the body.

21. The device according to any one of the preceding claims, wherein the body includes contoured surfaces adapted to be held by an operator while manipulating the device.

22. The device according to claim 21, wherein the contoured surfaces are texturized for improved gripping by the operator.

23. The device according to any one of the preceding claims, wherein the fluid inflow end of the body is releasably couplable with the needle.

24. The device according to any one of the preceding claims, wherein the device includes a cap that is configured to cover the needle while not in use.

25. The device according to any one of the preceding claims, wherein the device is adapted to sample fluid from an anatomical feature of the subject selected from the group including: a blood vessel, a cyst, an abscess and a blister.

26. The device according to any one of the preceding claims, wherein the needle size is 21 or 23 gauge.

27. The device according to any one of the preceding claims, wherein the sampled fluid has a volume in the range of 0.3 mL to 20 mL.

28. The device according to any one of the preceding claims, wherein the sampled fluid has a volume in the range of 0.3 mL to 2 mL.

29. The device according to any one of the preceding claims, wherein the sampled fluid has a volume of about 1 mL.

30. A collecting receptacle releasably couplable with a device for sampling fluid from a subject, the collecting receptacle being adapted to maintain an interior of the collecting receptacle in fluid communication with the ambient environment while coupled to the fluid sampling device, to permit fluid to be passively collected into the collecting receptacle during fluid sampling.

31. The collecting receptacle according to claim 30, wherein the collecting receptacle has an opening and is couplable with the device such that the opening is not fully occluded by the coupling so as to maintain fluid communication between the interior of the collecting receptacle and the ambient environment.

32. The collecting receptacle according to claim 30 or claim 31, wherein one or both of the device and collecting receptacle include an engagement surface that urges the collecting receptacle and device apart when there is relative rotational movement between the components, thereby decoupling the collecting receptacle and the device.

33. The collecting receptacle according to claim 32, wherein the engagement surface includes a contoured edge on a portion of the collecting receptacle that is configured to abut against a contoured edge of the device when coupled thereto, and when one of the collecting receptacle and the device is rotated relative to the other of the device and the collecting receptacle, the relative movement between their abutting portions urges the components apart.

34. The collecting receptacle of claim 33, wherein the abutting portion of the collecting receptacle includes at least one winged portion having an edge that is shaped to abut against the contoured edge of the body, and wherein the collecting receptacle is rotatable by an operator grasping and rotating the winged portion.

35. The collecting receptacle of any one of claims 30 to 34, wherein at least part of the collecting receptacle is compliant to allow the sampled fluid to be expelled therefrom by squeezing or compressing.

36. The collecting receptacle according to any one of claims 30 to 35, wherein the collecting receptacle includes an indicator to allow the amount of fluid collected to be ascertained during fluid collection.

37. The collecting receptacle according to any one of claims 30 to 36, wherein the collecting receptacle has a volume capacity in the range of 0.3 mL to 20 mL.

38. The collecting receptacle according to any one of claims 30 to 37, wherein the collecting receptacle has a volume capacity in the range of 0.3 mL to 2 mL.

39. The collecting receptacle according to any one of claims 30 to 38, wherein the collecting receptacle has a volume capacity of about 1 mL.

40. A method for sampling fluid from a subject, the method including the steps of:
(a) providing a sampling device including a body having a fluid outflow end and a needle at a fluid inflow end of the body;
(b) inserting the needle into the subject;
(c) sampling fluid by allowing fluid to flow from the subject, through the needle, and into the body through the fluid inflow end for collection at the fluid outflow end in a collecting receptacle; and
(d) removing the needle from the subject,
wherein the fluid is passively collected into the collecting receptacle during fluid sampling through an arrangement between the fluid outflow end of the body and collecting receptacle that maintains an interior of the collecting receptacle in fluid communication with the ambient environment.

41. The method according to claim 40, further including, after providing the sampling device, the step of:
coupling the collecting receptacle to the body at the fluid outflow end, wherein the coupling is such that the interior of the collecting receptacle is in fluid communication with the ambient environment.

42. The method according to claim 40 or claim 41, wherein the needle is inserted to penetrate the subject to a needle depth sufficient for the sampling device to self-anchor during fluid sampling.

43. The method according to any one of claims 40 to 42, further including, after inserting the needle into the subject, the step of:
checking for fluid flash-back in the body to confirm good fluid access, wherein fluid flash-back is visible through at least a transparent portion of the body during fluid sampling.

44. The method according to claim 43, wherein, in the absence of fluid flash-back, the method includes the steps of:
removing the needle from the subject; and
repeating the inserting, sampling and removing steps.

45. The method according to any one of claims 40 to 44, including the step of:
compressing tissue of the subject near the needle to increase fluid flow into the needle.

46. The method according to any one of claims 40 to 45, further including the step of:
checking the fluid level in the collecting receptacle during fluid sampling by inspecting an indicator on the collecting receptacle.

47. The method according to claim 46 when appended to claim 41, wherein when the fluid level is sufficient, the method includes the step of:
decoupling the collecting receptacle by rotating it with respect to the body.

48. The method according to claim 47, further including, after decoupling the collecting receptacle, the step of:
squeezing or compressing the decoupled collecting receptacle to expel the sampled fluid into a device, collection vessel or pathology tube.

49. The method according to claim 47 or claim 48, further including, after decoupling the collecting receptacle, the steps of:
coupling a collecting receptacle to the body at the fluid outflow end; and
repeating the sampling step.

50. The method according to any one of claims 40 to 49, wherein the needle is inserted into an anatomical feature selected from the group including: a blood vessel, a cyst, an abscess and a blister.

51. The method according to any one of claims 40 to 50, wherein the sampled fluid has a volume in the range of 0.3 mL to 20 mL.

52. The method according to any one of claims 40 to 51, wherein the sampled fluid has a volume in the range of 0.3 mL to 2 mL.

53. The method according to any one of claims 40 to 52, wherein the sampled fluid has a volume of about 1 mL.

54. A kit including:
a. a sampling device including a body having a fluid inflow end and a fluid outflow end, and a needle at the fluid inflow end that is adapted to penetrate a subject to sample fluid; and
b. one or more collecting receptacles,
wherein the body has a body portion at the fluid outflow end that is couplable with the one or more collecting receptacles and configured such that an interior of the one or more collecting receptacles remains in fluid communication with the ambient environment to permit fluid to be passively collected into the one or more collecting receptacles during fluid sampling.

55. The kit according to claim 54, wherein the one or more collecting receptacles have a volume capacity selected from one of: in the range of 0.3 mL to 20 mL, in the range of 0.3 mL to 2 mL, and about 1 m L.

56. The kit according to claim 54 or claim 55, wherein the kit includes two or more collecting receptacles and each collecting receptacle has a different volume capacity.

57. The kit according to any one of claims 54 to 56, wherein the one or more collecting receptacles includes an anticoagulant.

58. The kit according to claim 57, wherein the anticoagulant is selected from one of the group including: sodium heparin, potassium oxalate, ethylene diamine tetraacetic acid (EDTA), sodium citrate, acid citrate dextrose (ACD), sodium polyanethol sulfonate (SPS) and thrombin.

59. The kit according to any one of claims 54 to 58, wherein the kit includes two or more collecting receptacles and each collecting receptacle includes a different anticoagulant.

60. A method for sampling fluid from a subject using the kit according to any one of claims 54 to 59.
